(19) United States
(12) Patent Application Publication
(10) Pub. No.: US 2026/0112280 A1
(43) Pub. Date: Apr. 23, 2026

(54) MEDICAL APPLICATION GAME GENERATOR FOR PRESCRIPTION INTELLIGENCE EVALUATION

(71) Applicants: Stephen Farrow, Biloxi, MS (US); Aljon Viray, Sunnyvale, CA (US); Rossette Farrow, Biloxi, MS (US)

(72) Inventors: Stephen Farrow, Biloxi, MS (US); Aljon Viray, Sunnyvale, CA (US); Rossette Farrow, Biloxi, MS (US)

(21) Appl. No.: 19/415,252

(22) Filed: Dec. 10, 2025

Related U.S. Application Data
(63) Continuation-in-part of application No. 18/401,577, filed on Dec. 31, 2023, now abandoned.
(60) Provisional application No. 63/436,921, filed on Jan. 4, 2023.

(51) Int. Cl. G09B 5/02; G16H 10/60

(52) U.S. Cl. CPC G09B 5/02; G16H 10/60

(57) ABSTRACT

A software program application that elicits, collects, corrects, and records patient responses to illness-diagnosis, prescribed medical-treatment, and anatomical and other relevant health-elements matching scenarios presented in crossword, scrolling matching, scrolling-capture, adventure-construction, adventure-capture, ballistic-target (shooter), and other video game formats, without-or-with virtual reality and without-or-with avatars is provided. Game application criteria for success may be based on diagnostic, prescribed medication/management, and optional anatomical or other related information that the application imports securely in compliance with HIPAA requirements from the Patient-Player's web health portal.

### CROSS REFERENCE TO RELATED APPLICATIONS

This application claims priority to and the benefit of the filing date of U.S. application Ser. No. 18/401,577, filed on Dec. 31, 2023 which in turn claims priority to and the benefit of the filing date of U.S. Provisional Application No. 63/436,921, filed on Jan. 4, 2023, the contents of both of which are hereby incorporated by reference in their entirety.

### BACKGROUND OF THE INVENTION

This invention relates to a software program application and system to motivate patients to study the management of their illnesses, to increase patient understanding of and adherence to prescriptions, and to help clinicians optimize illness management and patient instruction.

Physicians prescribe medications to manage illness in patients. One of the most common preventable reason that medications fail in treating their target illnesses is that patients do not accurately follow prescription instructions. This failure is known as nonadherence. Medication regimens can be complex and confusing. Patients may misunderstand and/or have difficulty following medication instructions because of illness-related problems, limited education, poor vision, or competing priorities. As the number of prescribed medications increases, it can become more difficult for the patient to follow the instructions for any individual medication, including timing, dose, and relation to meals, activities, and other medications. Further, the patient may not understand or may forget important instructions on how to recognize or respond to adverse medication reactions. Numerous strategies, devices, and applications have been developed to help patients time medications correctly, with varying degrees of success. A novel solution that can help the patient develop and practice at-least minimum understanding of that patient's illnesses and the relationships of prescribed medications and treatments to the illnesses in a fun, engaging way would increase adherence to prescriptions and improve communication with the physician and the rest of the medical/health team when adverse events threaten. Such a novel application would also help the patient understand and effectively participate in developing the medical/health team's management plan. Improved health and reduced money-and time-costs for managing illness would result.

### BRIEF SUMMARY OF THE INVENTION

A Medical Information Gaming System is provided. In some embodiments, and in accordance with a first aspect, the system may include a multi-part software program application (application) that may utilize a programmable device that retrieves, stores, and processes data (eg, smart-phone, tablet, computer) to engage the Patient-User of the application as a Game-Player (hereafter, the “Player”) in playing a learning game. The game presents multiple options for the Player to practice linking (by matching or other methods) information on diagnosed illnesses, associated prescribed medications and treatments and possible side-effects, and the affected body-organs and related issues. The game application may use a variety of platforms, including basic crossword and puzzle-platforms, intermediate-level vertical-and side-scrolling, three-dimensional platform-adventure, and other formats. Platforms may be exercised without or with virtual reality, and without or with avatars. The application may be divided into multiple stages of varying complexity. In some embodiments, the application rewards the Player with points and/or stage-advancement for correct linking of a diagnosis, prescribed medication, and optional images, text, and/or sounds associated with relevant anatomy, functions, or other important features. Preferably, to progress to the next level, all stages in the previous level must have been completed. In some embodiments, if a stage is answered incorrectly or the Player provides other incorrect input, the application may announce the error to the Player, may give the Player the correct answer, may send the Player back to the beginning of the stage, and/or may charge one of a finite number of game turns in the process. Once a Player has exhausted available turns in a given stage, the game may terminate, and the Player may be encouraged to resume the game from the end of the last successfully-completed level.

Depending on the particular game, the application may use color, sound, motion, text, speech, and/or avatars to engage and motivate the Player in the entertaining and enjoyable matching effort. The application preferably advances the Player from simple to more-complex engagements as the Player's learning (as documented by successive passing game scores) increases. Scores and other Player information may be archived for review and may be used to guide prescription and coaching by the medical/health team.

### DETAILED DESCRIPTION AND BEST MODE OF IMPLEMENTATION

In some embodiments, the Medical Information Gaming System (also called medical application game generator for prescription intelligence evaluation) may include a software program application that exercises Health Insurance Portability and Accountability Act (HIPAA)-compatible security to import pertinent health portal data (diagnosis, treatment, anatomy, etc) and assemble updated game-play scenarios. Preferably, the software program application follows single-and multi-armed entry-detection/correction feedback loops to evaluate and correct Player in-game matching responses. The Player's game decisions may be immediately scored and recorded. Accurate responses may continue the game session. Successful completion of one stage advances the Player to the next stage, and successful completion of all stages in a level advances the Player to the next level. Inaccurate responses may interrupt the game, preferably to provide the Player with the correct response and related information. Depending on the nature of any incorrect responses, the Player may have the option to replay the incorrect stage or to progress to the next stage in a given level of the game. Once the limit of incorrect responses is reached, the active game session may terminate, and the Player may be encouraged to start a new game at the current or an earlier level. Although the Player has some flexibility in selecting the level and stage to engage, the game strongly recommends that the Player engage the game a level appropriate to the Player's previously-demonstrated/documented performance and knowledge. In some embodiments, when the Player interrupts play between stages, the application may check the health portal for new patient information. If new information is found, the application may reset the game parameters to reflect the new information. The game may then allow play to start at the newly-appropriate level. Depending on the information updated, this level may be lower than that of the last successfully-completed level; the application would inform the Player of the reason for this adjustment.

In some embodiments, Player responses may entail selecting the appropriate matching objects. For example, these can be the correct word to continue the acrostic, the correct token to match additional tokens operative in the game, the appropriate doorway or other path, or the correct projectile/launcher/target match, and so on. In preferred embodiments, at any level the Player may have one or more opportunities, or stages, in which to correctly match diagnostic and treatment elements, and possibly optional anatomical or other relevant elements. The Player may then progress to the next level. Information on use-volume and response-statistics may be recorded by the application and may be transmitted securely back to the health portal via a HIPAA-compliant web-scraper. Preferably, the medical/health team may review the data for number of instances and durations of play and correct answers and focused areas of expertise or shortfall. The medical/health team may then adjust management of the illness; the adjustments may be reflected in the health portal. The application's web-scraping and automated robotic processes may leverage the health portal data to update the game's terms for success. The medical/health team may choose to further educate and coach the Player through virtual or in-person communication.

The application was developed using the Unity multi-platform game engine. Unity uses the C# programming language. However, any other suitable programming method may be used by the system. In preferred embodiments, the software application program may run on iOS (Apple devices, such as iPhones), Android, standalone PC (an “.exe” file, for example), WebGL/HTML5 (Website-based), and any other platform/operating system that Unity supports.

In some embodiments, a Medical Information Gaming System may include a Player-motivating education computer software program application with graphic user interfaces for programmable data-storage and processing devices such as smart phone, tablet, computer hardware, or other interface capable of facilitating multiple game-types.

In preferred embodiments, a computer software program application may require a Player to provide input (via a programmable interface, data-storage and processing device) describing correct matching among medical diagnosis, prescribed-treatment, and related anatomical and/or other elements in a variety of game-play formats.

In some embodiments, current diagnosis and management and optional anatomical or other pertinent element-matching for the gameplay experience provided to a Player may be informed by Health Insurance Portability and Accountability Act-(HIPAA)-compliant importation of diagnostic and management plan data from the health portal into the application's ground-criteria storage database.

In some embodiments, the system may utilize a game-strategy of matching medical diagnosis, prescribed management, and (optional) related anatomical or other elements based on ground-criteria data.

In some embodiments, the system may utilize a variety of game formats, including but not limited to: A. Crossword; B. Scrolling platform object-match; C. Scrolling platform object capture; D. Adventure-construction; E. Adventure-capture; and F. Ballistic target (ie, shooter)/object capture. Embodiments may include virtual reality settings and/or avatars.

In some embodiments, Player success entails completion of each gaming stage through correct matching of diagnosis, medication/treatment, and other optional elements such as relevant anatomy according to the particular game's rules.

In some embodiments, the system may utilize an in-application check system rewarding the Player for correct matches, educating the Player on incorrect matchings, and displaying continuous in-stage, end-of-stage or-level, and/or cumulative-play scores.

In some embodiments, the system may utilize a mechanism to store downloaded health portal data, Player activity, and scoring and performance details in tabular, graphic, or various summary formats.

In some embodiments, the system may utilize a transmission mechanism to deliver application statistics of a Player to a health portal by virtual or physical connection or by other means.

In some embodiments, the system may utilize a communication-transmission mechanism that alerts the medical/health team to convey enhanced instruction to the Player by way of the health portal and the application and gaming hardware, or by direct virtual or in-person means.

What is claimed is:

1. A computer-implemented method for patient-specific educational gameplay, comprising:
adjusting the ground criteria for success based on updates found in the health portal and resuming gameplay at an appropriately determined level.

2. The method of claim 1, wherein presenting the gameplay scenarios includes one or more of: crossword; scrolling platform object-match; scrolling platform object capture; adventure-construction; adventure-capture; and ballistic target object capture.

3. The method of claim 1, further comprising presenting the gameplay scenarios in virtual reality and/or with avatars.

4. The method of claim 1, wherein receiving the player inputs includes one or more of: selecting a word to continue an acrostic; choosing tokens to match additional tokens operative in the game; selecting an appropriate doorway or path; and selecting a projectile, launcher, and target match.

5. The method of claim 1, wherein awarding scores comprises displaying continuous in-stage scoring, end-of-stage or end-of-level scoring, and cumulative-play scoring, and rewarding points and/or stage advancement for correct matching.

6. The method of claim 1, wherein, upon an incorrect match, the method further comprises announcing the error, presenting the correct answer, returning the player to the beginning of the stage, and/or deducting one of a finite number of turns.

7. The method of claim 1, further comprising terminating an active game session when a limit of incorrect responses or turns is reached, and prompting the player to start a new game at a current or earlier level.

8. The method of claim 1, further comprising, upon player interruption between stages, checking the health portal for new patient information, resetting game parameters to reflect any new information, and allowing play to restart at a newly appropriate level, including informing the player when the level is lowered due to updated information.

9. The method of claim 1, wherein securely importing the patient health portal data and transmitting player activity and performance details are performed via a HIPAA-compliant web-scraper and automated robotic processes.

10. The method of claim 1, wherein the programmable device executes a software application implemented using the Unity multi-platform game engine in C#, and the application runs on one or more of: iOS, Android, standalone PC executable, and WebGL/HTML5.

11. A medical information gaming system, comprising:
in tabular, graphic, or summary formats;

12. The system of claim 11, wherein use-volume and response statistics transmitted to the health portal are reviewed by a medical or health team to adjust illness management, the adjustments are reflected in the health portal, and the data import module and game engine update the criteria for success based on the adjustments.

13. The system of claim 11, wherein the game engine employs one or more of color, sound, motion, text, and speech to engage and motivate the player, and advances from simpler to more complex scenarios based on successive passing game scores.

14. The system of claim 11, wherein the transmission module comprises a communication-transmission mechanism that alerts the medical or health team to convey enhanced instruction to the player via the health portal and the application and gaming hardware, or via direct virtual or in-person means.

15. A computer-implemented method comprising:
generating a clinician alert signal; and

16. The method of claim 15, wherein the clinician alert signal includes one or more of: player identification, stage or level reached, number of incorrect responses, cumulative score, and specific areas of knowledge deficiency.

17. The method of claim 15, wherein the enhanced instruction comprises updated prescription coaching, additional educational content, or scheduling of a virtual or in-person consultation.

18. The method of claim 15, wherein the clinician alert signal is generated automatically when a limit of incorrect responses or turns is reached, or when gameplay is terminated due to nonadherence.

19. The method of claim 15, wherein the clinician alert signal is transmitted concurrently with updated gameplay parameters to reset the player's level based on new health portal data.
